(19) United States
(12) Patent Application Publication
(10) Pub. No.: US 2025/0255519 A1
(43) Pub. Date: Aug. 14, 2025

(54) CONTROL OF A LIGHT SOURCE OF A PULSE OXIMETER

(71) Applicant: Loewenstein Medical Technology S.A., Luxembourg (LU)

(72) Inventor: Farzad SAMIE, Karlsruhe (DE)

(21) Appl. No.: 19/048,127

(22) Filed: Feb. 7, 2025

(30) Foreign Application Priority Data
Feb. 13, 2024 (DE) 102024104004.1

(51) Int. Cl. A61B 5/1455; G16H 40/63

(52) U.S. Cl. CPC A61B 5/14551; G16H 40/63

(57) ABSTRACT

A method for controlling a light source of a pulse oximeter comprises:
receiving a sensor signal generated by a light sensor for detecting a light component transmitted and/or reflected by a body part on irradiation with light from the light source;
receiving a current value of at least one control parameter for controlling a brightness and/or color of the light source;
determining a scaling factor from a plot of an amplitude of the sensor signal against time, taking into account a target value for the amplitude;
determining a new value for the at least one control parameter by multiplying the current value by the scaling factor;
applying the new value to the at least one control parameter.

### CROSS-REFERENCE TO RELATED APPLICATIONS

The present application claims priority under 35 U.S.C. § 119 of German Patent Application No. 102024104004.1, filed Feb. 13, 2024, the entire disclosure of which is expressly incorporated by reference herein.

### BACKGROUND OF THE INVENTION

### 1. Field of the Invention

The invention relates to a method for controlling a light source of a pulse oximeter. The invention further relates to a control unit, a computer program and a computer-readable medium for carrying out the method and to a pulse oximeter.

### 2. Discussion of Background Information

A pulse oximeter is generally a device for noninvasive determination of arterial oxygen saturation by measurement of light absorption or light remission as blood-perfused body tissue is being transilluminated. Such a pulse oximeter can also be used for monitoring pulse rate.

Pulse oximeters are increasingly being used in wearable, battery-operated applications. For example, a pulse oximeter may be attached to a patient during emergency transport and remain with said patient during transfer between different hospital departments. Furthermore, pulse oximeters may be used as plug-in modules for multiparameter patient monitors with a limited energy budget. Such applications mean that there is an increasing demand for pulse oximeters with a relatively low power consumption.

In view of the foregoing, it would be advantageous to have a method, by means of which the power consumption of a pulse oximeter, in particular a battery-operated pulse oximeter, can be reduced without any appreciable loss of quality. It can be considered to be a further object of the invention to provide a control unit, a computer program and a computer-readable medium for carrying out such a method and a corresponding pulse oximeter.

### SUMMARY OF THE INVENTION

In a first aspect the invention provides a method for controlling an (electrical) light source of a pulse oximeter. The pulse oximeter comprises in addition to the light source a light sensor designed to convert a light component transmitted and/or reflected by a body part on irradiation with light from the light source into an (electrical) sensor signal. The method comprises: receiving the sensor signal and a current value of at least one control parameter for controlling a brightness and/or color of the light source; determining a scaling factor from a plot of an amplitude of the sensor signal against time, taking into account a target value for the amplitude; determining a new value for the at least one control parameter by multiplying the current value by the scaling factor; applying the new value to the at least one control parameter.

The method allows saving of power in operation of the pulse oximeter, for example by reducing the current flowing through the light source, referred to as light-source current hereinafter, so long as the quality of the photoplethysmogram (PPG for short) from the sensor signal is still sufficient and the necessary accuracy is still maintained, but increasing said current if relatively strong noise occurs or if the quality of the sensor signal generally becomes worse.

In order to be able to ensure a sufficient quality when determining oxygen saturation, the amplitude of the pulsatile component of the sensor signal in the PPG, also referred to as AC component, should not fall below a specific value.

In general, the amplitude of the sensor signal is approximately linear to the light-source current in every patient. This is because physiological factors influencing the absorption of light by blood-perfused body tissue, such as finger thickness or skin color, do not change significantly during a measurement.

The AC component can be made to lie within the desired range by, for example, appropriately adjusting the light-source current during operation with the aid of the method. At the same time, excessively large and/or excessively frequent adjustments should be avoided.

The method may be computer-implemented.

The steps of the method may be carried out continuously, i.e., repeated cyclically, in operation of the pulse oximeter. In other words, the value of the at least one control parameter may be updated periodically at specific time intervals, for example of 0.01 s, 0.1 s, 1 s or 10 s, with the aid of the method.

“Sensor signal” can be understood to mean an analog or digital electrical signal. “Amplitude” can be understood to mean, in particular, an amplitude of the AC component of the sensor signal.

“Control parameter” can be understood to mean, for example, one of the following parameters: an electric current which flows or is to flow through the light source (e.g., between 3 mA and 50 mA); a voltage which is applied or is to be applied to the light source; an adjustable series resistance upstream of the light source; a frequency at which the light source is switched on and switched off; a time ratio between switch-on phases, in which the light source is switched on, and switch-off phases, in which the light source is switched off. For example, the frequency may be a clock frequency and/or the time ratio may be a duty cycle in the context of a pulse width modulation of the light source. The current value of the at least one control parameter may be a set and/or measured and/or estimated value.

“Color” can be understood to mean a wavelength or wavelength range of an electromagnetic spectrum. Different colors can be distinguished from one another in terms of their wavelength or wavelength range.

“Target value” can be understood to mean a fixed or variable desired value of the amplitude of the sensor signal. For example, the variable target value may be varied during operation depending on a (measured or estimated) change in specific physiological factors of the particular patient. The target value may be, for example, between 2 nA and 3 nA, preferably 2.5 nA.

The new value may be identical to the product of the current value and the scaling factor or it may be a value determined on the basis of the product of the current value and the scaling factor. The applying of the new value allows adjustment of the light source in terms of its brightness and/or color according to the new value (instead of the current value) of the at least one control parameter.

In a second aspect the invention provides a control unit. The control unit comprises elements configured to carry out the method described above and below.

The elements may generally comprise hardware and/or software modules. In particular, the means may comprise a processor configured to carry out the (computer-implemented) method. In addition, the elements may comprise a memory and/or a data communication interface for wireless and/or wired data communication with peripheral devices, for example a smartphone, a smartwatch, a tablet, a laptop, a PC or a ventilator. Alternatively, the control unit may be solely implemented as hardware, for example in the form of an ASIC component (ASIC=application-specific integrated circuit) or FPGA component (FPGA=field-programmable gate array).

It should be noted that features of the method described above and below may also be features of the control unit (and vice versa).

In a third aspect the invention provides a pulse oximeter. The pulse oximeter comprises a light source, a light sensor and a control unit, as described above and below. The light sensor is designed to convert a light component transmitted and/or reflected by a body part on irradiation with light from the light source into an (electrical) sensor signal.

The pulse oximeter, for example the control unit thereof, may be designed to determine an oxygen saturation (sO₂) and/or a pulse from the sensor signal by absorption spectroscopy.

“Light source” can be understood to mean, for example, a light-emitting diode, a laser diode, an incandescent lamp or a combination of at least two of these examples. In particular, the light source may be designed to emit light in at least two different predetermined wavelength ranges, for example red light, (near-) infrared light or green light. Examples of suitable wavelength ranges are 660 nm, 750 nm to 850 nm, and 905 nm to 940 nm.

“Light sensor” can be understood to mean, for example, a photodiode, a photocell, a CMOS sensor (CMOS=complementary metal-oxide-semiconductor), a CCD sensor (CCD=charge-coupled device) or a combination of at least two of these examples.

“Pulse oximeter” can also be understood to mean a CO-oximeter.

The pulse oximeter may be designed, for example, as a clip for attachment to the body part, for example a finger, an earlobe or a wrist.

The pulse oximeter may be designed such that the light source and the light sensor can be arranged on the same side of the body part and/or on mutually opposite sides of the body part.

In further aspects the invention provides a computer program and a computer-readable medium on which the computer program is stored.

The computer program comprises commands which cause a processor—for example a processor of the control unit described above and below—to carry out the method described above and below when the processor executes the computer program.

The computer-readable medium may be a volatile or non-volatile data memory. For example, the computer-readable medium may be a hard disk, a USB storage device (USB=universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communication network which enables program code to be downloaded (e.g., via the Internet), or a cloud.

It should be noted that features of the method described above and below may also be features of the computer program and/or the computer-readable medium (and vice versa).

Various embodiments of the invention are described hereinafter. These embodiments should not be understood as restricting the scope of the invention.

According to one embodiment, an average may be determined from the plot of the amplitude against time. The average may then be used for determining the scaling factor. “Average” can be understood to mean, for example, an arithmetic mean, geometric mean, root mean square or exponential smoothing average. The average can be understood as a quality index (“QI” for short) in relation to the sensor signal.

According to one embodiment, the scaling factor may be determined by forming a quotient from the average and the target value. The scaling factor may be identical to the quotient or it may be a value determined on the basis of the quotient. The quotient may be formed by dividing the target value by the average or vice versa.

According to one embodiment, the average may be a moving average. The moving average may be a simple or weighted moving average. This allows effective smoothing of the sensor signal before further processing thereof. The moving average acts like a low-pass filter. In principle, the moving average may be determined by sampling the sensor signal section-wise in a window in multiple consecutive time steps. In each of the time steps, an average may be calculated from the values of the sensor signal in the respective window, and the window may then be shifted such that it partly overlaps with the window of the last time step. For example, the window between (immediately) consecutive time steps may be shifted such that the last value in the last-sampled section of the sensor signal is deleted from the window and the first value in the currently sampled section of the sensor signal, i.e., the first value following the last-sampled section, is included in the window. The average may then be recalculated from the values of the window updated this way. In addition, the values in the window may be appropriately weighted. The width of the window may be fixed, i.e., constant, or variable, for example the window may vary from time step to time step. In other words, the window in one of the time steps may have a different width than in at least one other of the time steps.

According to one embodiment, the sensor signal may be received in multiple consecutive time steps. In this case, the average may be determined in each of the time steps using an actual value of the amplitude of the sensor signal received in the respective time step and/or using an earlier (or at least one earlier) average determined in an earlier time step preceding (e.g., immediately preceding) the respective time step. This allows effective smoothing of the sensor signal before further processing thereof. The (moving) average acts like a low-pass filter.

According to one embodiment, the method may be carried out in multiple consecutive time steps, wherein, in each of the time steps, the sensor signal and the current value may be received, the average, the scaling factor and the new value may be determined and the new value may be applied.

According to one embodiment, the actual value and the earlier average (or the earlier averages) may be weighted differently. This allows flexible adaptation of smoothing to different operating conditions.

According to one embodiment, the average may be determined in each of the time steps according to the following equation:

$$QI = α * A + ( 1 - α ) * {QI}_{alt} .$$

Here, QI may be the average in the respective (current) time step, A may be the actual value, QIₐₗₜ may be the earlier average and α may be a weighting factor. The weighting factor may be, for example, a value between 0 and 1. The weighting factor may be, in particular, an empirical value. The weighting factor may be identical, i.e., constant, in each of the time steps or variable, for example it may vary from time step to time step. In other words, the weighting factor in one of the time steps may differ from the weighting factor in at least one other of the time steps.

According to one embodiment, the applying of the new value may comprise: determining a deviation value indicating a deviation of the new value from the current value; determining an adjustment value using the deviation value and an assignment rule, by means of which possible deviation values are each assigned an adjustment value; determining an adjusted new value using the current value and the adjustment value, in particular by adding up the current value and the (positive or negative) adjustment value; applying the adjusted new value to the at least one control parameter. In other words, the magnitude of the new value may be appropriately altered before application thereof to the at least one control parameter. This makes it possible, for example, to avoid excessively large and/or excessively frequent jumps in setting the at least one control parameter. “Assignment rule” can be understood to mean, for example, a mathematical function or a lookup table. The assignment rule may be stored, for example, in a memory of the control unit described above and below.

According to one embodiment, the assignment rule may be a sigmoid function or be based on a sigmoid function. “Sigmoid function” can be generally understood to mean a function with an S-shaped curve. The sigmoid function may additionally comprise a linear or approximately linear section. Such a section may be relevant, inter alia, to the stability of the method.

According to one embodiment, the assignment rule may be defined as follows:

$$f ( Δ ) = \frac{2 \times S}{1 + {2}^{- Δ}} - S .$$

Here, f(Δ) may be the adjustment value, Δ may be the deviation value and S may be a maximum permissible magnitude of the adjustment value. During operation of the pulse oximeter, the magnitude may be constant or variable, for example depending on the current operating conditions. The magnitude may also be referred to, for example, as (maximum) step size.

According to one embodiment, an approximation P for the term 2^{-Δ} based on a series expansion, preferably a Taylor series and particularly preferably a Maclaurin series, may be determined. In this case, the assignment rule may be defined as follows:

$$f ( Δ ) = \frac{2 \times S}{1 + P} - S .$$

This allows improved computational efficiency compared to embodiments where the term 2^{-Δ} is calculated instead of P. Thus, the power consumption of the pulse oximeter can be reduced further. Moreover, this facilitates the implementation of the method as hardware and/or software.

According to one embodiment, the approximation P may be defined as follows:

$$if - Δ \geq 0 , then P = 1 + {\sum }_{n = 1}^{N} \frac{{( k \times ( - Δ ) )}^{n}}{n !} and / or$$

$$if - Δ < 0 , then P = \frac{1}{1 + {\sum }_{n = 1}^{N} \frac{{( - k \times ( - Δ ) )}^{n}}{n !}} ;$$

Here, N may be a predetermined order of the series expansion and k may be a predetermined factor. For example, N may be a natural number between 1 and 10, preferably 5, and/or k may be a percentage between 0 and 1, preferably between 0.5 and 1.0 and particularly preferably 0.75. N and/or k may be, in particular, an empirical value/empirical values.

This allows an approximation that is particularly computationally efficient and/or particularly simple to implement, without noticeable impairment of the accuracy of the method.

According to one embodiment, the applying of the new value may comprise: determining a rounded value from the new value; applying the rounded value to the at least one control parameter. For example, the new value may be exactly rounded to one or two decimal places according to the convention for rounding. For example, the limit for the rounded value “1” may be 0.5 to 1.4 or 0.45 to 1.54. This can simplify further processing of the new value. “New value” in this context can also be understood to mean an adjusted new value, as described above.

According to one embodiment, the determining of the rounded value may comprise: doubling the new value; rounding the doubled value; halving the rounded doubled value. This allows a relevant reduction of decimal places without significant impairment of accuracy.

According to one embodiment, the pulse oximeter may further comprise a battery for supplying power to at least one electrical or electronic component of the pulse oximeter, in particular the light source, or the entire pulse oximeter. This enables the pulse oximeter to also be worn on the go.

According to one embodiment, the pulse oximeter may further comprise a display unit for displaying at least one value determined using the sensor signal, for example an oxygen saturation or a pulse.

### BRIEF DESCRIPTION OF THE DRAWINGS

Embodiments of the invention are described hereinafter with reference to the accompanying drawings. Neither the description nor the drawings should be understood as restricting the scope of the invention.

FIG. 1 shows a pulse oximeter according to one embodiment of the invention.

FIG. 2 shows a diagram illustrating an assignment rule for use in a method according to one embodiment of the invention.

The drawings are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

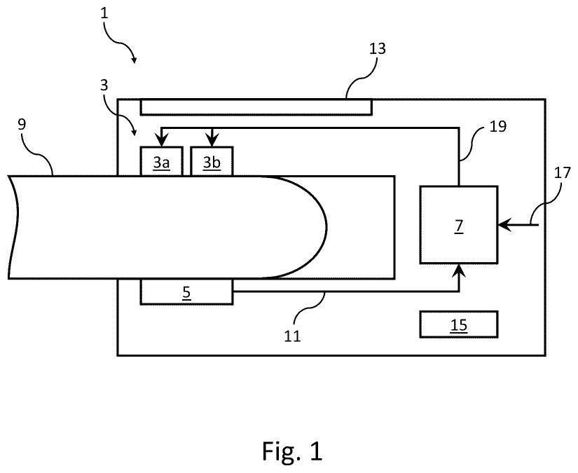

FIG. 1 shows a pulse oximeter 1 comprising an (electrical) light source 3, a light sensor 5 and a control unit 7. The light sensor 5 is designed to convert a light component transmitted and/or reflected by a body part 9, for example a finger, an earlobe or a similarly thin body part, on irradiation with light from the light source 3 into an (electrical) sensor signal 11.

In this example, the control unit 7 is designed to determine an oxygen saturation (sO₂) and/or a pulse from the sensor signal 11.

In addition, the pulse oximeter 1 may comprise a display unit 13 for displaying at least one value and/or at least one graph in relation to the sensor signal 11, in particular in relation to the oxygen saturation and/or the pulse. The display unit 13 may be arranged, for example, in the form of a display in and/or on a housing of the pulse oximeter 1.

It is convenient if the pulse oximeter 1 comprises a battery 15 for supplying power to the pulse oximeter 1. The pulse oximeter 1 can thus also be worn on the go.

The control unit 7 may be generally designed to alter the intensity and/or color of the emitted light by appropriate actuation of the light source 3. In this example, the light source 3 comprises a first light-emitting diode 3a for emitting light in a first wavelength range, for example 660 nm, and a second light-emitting diode 3b for emitting light in a second wavelength range different from the first wavelength range, for example 880 nm to 940 nm. The control unit 7 may be designed to switch on and switch off the light-emitting diodes 3a, 3b mutually alternatingly in operation of the pulse oximeter 1. Alternatively, the light source 3 may comprise just one light-emitting diode having a suitably variable wavelength range. Other types of light source such as laser diodes or incandescent lamps are also possible.

The light sensor 5 may comprise, for example, a photodiode, a photocell, a CMOS sensor, a CCD sensor or a combination of at least two of these examples.

The pulse oximeter 1 may be designed, for example, as a clip for attachment to the body part 9 and/or as a CO-oximeter.

In this example, the pulse oximeter 1 is designed such that the light source 3 and the light sensor 5 are arranged on mutually opposite sides of the body part 9 in operation of the pulse oximeter 1. The light sensor 5 thus mainly receives the light component transmitted by the body part 9 when the light source 3 illuminates the body part 9.

Alternatively, the pulse oximeter 1 may be designed such that the light source 3 and the light sensor 5 are arranged on the same side of the body part 9 in operation of the pulse oximeter 1.

The control unit 7 comprises means configured to carry out a specific method for controlling supply of power to the light source 3, as will be described in greater detail below. The means may comprise hardware and/or software modules. In particular, the means may comprise a memory and a processor. A computer program may be stored in the memory, and the processor may be configured to carry out the method by executing the computer program. In addition, the means may comprise a data communication interface for wireless and/or wired data communication with peripheral devices, for example a smartphone, a smartwatch, a tablet, a laptop, a PC or a ventilator.

The control unit 7 may also be solely implemented as hardware, for example in the form of an ASIC component or FPGA component.

The method, which may be computer-implemented, may comprise the following steps.

A first step comprises receiving in the control unit 7, for example in a corresponding hardware and/or software module of the control unit 7, the sensor signal 11 and a current value 17 of at least one control parameter for controlling the brightness and/or color of the light source 3. In this example, the at least one control parameter is an electric current flowing through the light source 3. Other control parameters are, however, also possible, for example a voltage applied to the light source 3. The current value 17 may be, for example, a set and/or measured and/or estimated value.

A second step comprises determining a scaling factor from a plot of an amplitude of the sensor signal 11 against time, taking into account a target value for the amplitude.

A third step comprises determining a new value for the at least one control parameter by multiplying the current value 17 by the scaling factor. The new value may be, for example, equal to the product of the current value 17 and the scaling factor.

A fourth step comprises applying the new value to the at least one control parameter. For example, a control signal 19 for controlling the light source 3 may be generated according to the new value.

The method allows saving of power in operation of the pulse oximeter 1, for example by reducing the current flowing through the light source 3, so long as the quality of the photoplethysmogram (PPG for short) from the sensor signal 11 is still sufficient and the necessary accuracy is still maintained, but increasing said current if relatively strong noise occurs or if the quality of the sensor signal 11 generally becomes worse.

For example, in the second step, an average QI may be determined from the plot of the amplitude against time. The scaling factor may then be determined using the average QI and the target value, in particular as the quotient from the average QI and the target value.

It is possible that the sensor signal 11 is received in multiple consecutive time steps. In this case, the average QI may be determined in each of the time steps from an actual value of the amplitude of the sensor signal 11 received in the respective (current) time step and from at least one earlier actual value of the amplitude of the sensor signal 11 received in at least one earlier time step preceding the current time step.

Alternatively or additionally, the average QI may be determined in each of the time steps using the actual value of the amplitude of the sensor signal 11 received in the respective time step and using an earlier (or at least one earlier) average QIₐₗₜ determined in an earlier time step preceding (e.g., immediately preceding) the respective time step. Such a moving average allows effective smoothing of the sensor signal 11 before further processing thereof. The moving average acts like a low-pass filter.

Such a low-pass filter can be very simple to implement when the average QI is determined in each of the time steps according to the following equation:

$$QI = α * A + ( 1 - α ) * {QI}_{alt} .$$

Here, A is the actual value of the amplitude in the current time step and α is a weighting factor.

The weighting factor α may be, for example, any value between 0 and 1. The weighting factor α may be identical, i.e., constant in each of the time steps or variable, for example it may vary from time step to time step depending on changing operating conditions. In other words, the weighting factor α in one of the time steps may differ from the weighting factor α in at least one other of the time steps.

In addition, before the new value is applied to the at least one control parameter, it may be appropriately rounded, for example by doubling the new value, rounding the doubled value and lastly halving the rounded doubled value.

Conveniently, the at least one control parameter is set to the new value only if it differs significantly from the current value. In this case, it is possible that the average QI for the next time step is equal to the current actual value of the amplitude, while in other cases the average QI is calculated according to the aforementioned equation.

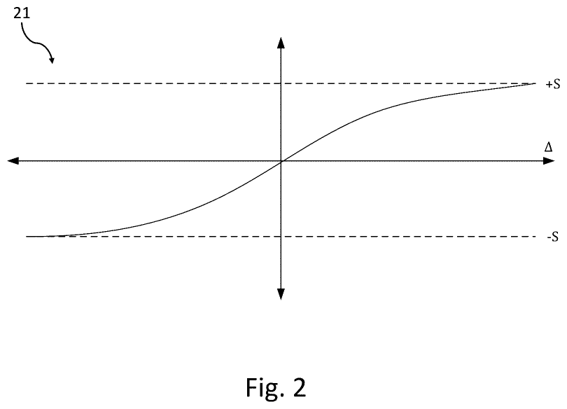

Optionally, a deviation value Δ indicating a deviation of the new value from the current value 17 may be determined, for example by subtracting the new value from the current value 17 (or vice versa). Using the deviation value Δ and a suitable assignment rule 21 (see FIG. 2), a positive or negative adjustment value may then be determined. The adjustment value may be used to calculate an adjusted new value, for example by adding up the current value 17 and the adjustment value. The adjusted new value may then be applied—as the new value—to the at least one control parameter.

The assignment rule 21 may be stored, for example, in the form of a mathematical function or a lookup table in the memory of the control unit 7.

In particular, the assignment rule 21 may be a mathematical function based on a sigmoid function. Such a function may be defined, for example, as follows:

$$f ( Δ ) = \frac{2 \times S}{1 + {2}^{- Δ}} - S ,$$

where f(Δ) is the adjustment value and S is a maximum permissible magnitude of the adjustment value (that is variable or constant in operation of the pulse oximeter 1) or a maximum step size. This can avoid undesirable fluctuations and/or jumps in the value of the at least one control parameter (and hence the amplitude of the sensor signal 11) between consecutive time steps.

In order to reduce computational cost, especially when using a microcontroller in the control unit 7, one option may be to calculate an approximation P instead of the term 2^{-Δ} in the function, such that it reads:

$$f ( Δ ) = \frac{2 \times S}{1 + P} - S .$$

The approximation may be performed, for example, according to the following equation:

$${2}^{- Δ} \approx apprx ( - Δ ) = \{ \begin{matrix}1 + {\sum }_{n = 1}^{N} \frac{{( k \times ( - Δ ) )}^{n}}{n !} , & - Δ \geq 0 \\ \frac{1}{1 + {\sum }_{n = 1}^{N} \frac{{( - k \times ( - Δ ) )}^{n}}{n !}} , & - Δ < 0\end{matrix}$$

Here, N is a predetermined order of the series expansion and k is a predetermined factor. For example, N may be a natural number between 1 and 10, preferably 5, and/or k may be a percentage between 0 and 1, preferably between 0.5 and 1.0 and particularly preferably 0.75. The number N influences the accuracy of approximation and the computational cost. For example, with N=5, a good compromise can be achieved between accuracy and computational cost.

Such a series expansion may also be referred to as a Maclaurin series. This allows an approximation that is particularly computationally efficient and/or particularly simple to implement, without noticeable impairment of the accuracy of the method.

As can be seen in FIG. 2, the assignment rule 21 may comprise a relatively long (approximately) linear section around the zero point, which is important for calculation stability.

Finally, it should be noted that terms such as “have”, “comprise”, “include”, “with”, etc., do not exclude other elements or steps, and indefinite articles such as “a” or “an” do not exclude a plurality.

Furthermore, it should be noted that features or steps described with reference to one of the embodiments above may also be used in combination with features or steps described with reference to other embodiments from among the embodiments above.

### LIST OF REFERENCE NUMERALS

1 pulse oximeter
3 light source
3a first light-emitting diode
3b second light-emitting diode
5 light sensor
7 control unit
9 body part
11 sensor signal
13 display unit
15 battery
17 current value
19 control signal
21 assignment rule
S maximum permissible magnitude
Δ deviation value

What is claimed is:

1. A method for controlling a light source of a pulse oximeter, the pulse oximeter comprising in addition to the light source a light sensor designed to convert a light component transmitted and/or reflected by a body part on irradiation with light from the light source into a sensor signal, wherein the method comprises:
receiving the sensor signal and a current value of at least one control parameter for controlling a brightness and/or color of the light source;
determining a scaling factor from a plot of an amplitude of the sensor signal against time, taking into account a target value for the amplitude;
determining a new value for the at least one control parameter by multiplying the current value by the scaling factor;
applying the new value to the at least one control parameter.

2. The method of claim 1, wherein an average is determined from the plot of the amplitude against time and the scaling factor is determined using the average.

3. The method of claim 2, wherein the scaling factor is determined by forming a quotient from the average and the target value.

4. The method of claim 2, wherein the average is a moving average.

5. The method of claim 2, wherein the sensor signal is received in multiple consecutive time steps, the average being determined in each of the time steps using an actual value of the amplitude of the sensor signal received in a respective time step and/or using an earlier average determined in an earlier time step preceding the respective time step.

6. The method of claim 5, wherein the actual value and the earlier average are weighted differently when determining the average.

7. The method of claim 5, wherein the average is determined in each of the time steps according to the following equation:
$$QI = α * A + ( 1 - α ) * {QI}_{alt} ;$$
where QI is the average in a respective time step, A is the actual value, QIₐₗₜ is the earlier average and α is a weighting factor.

8. The method of claim 1, wherein applying the new value comprises:
determining a deviation value (Δ) indicating a deviation of the new value from a current value;
determining an adjustment value using the deviation value (Δ) and an assignment rule, by means of which possible deviation values are each assigned an adjustment value;
determining an adjusted new value using the current value and the adjustment value;
applying the adjusted new value to the at least one control parameter.

9. The method of claim 8, wherein determining an adjusted new value using the current value and the adjustment value is effected by adding up the current value and the adjustment value.

10. The method of claim 8, wherein the assignment rule is a sigmoid function or is based on a sigmoid function; and/or the assignment rule is defined as follows:
$$f ( Δ ) = \frac{2 \times S}{1 + {2}^{- Δ}} - S ,$$
where f(Δ) is the adjustment value, Δ is the deviation value (Δ) and S is a maximum permissible magnitude (S) of the adjustment value.

11. The method of claim 10, wherein an approximation P for the term 2^{-Δ} based on a series expansion, is determined and the assignment rule is defined as follows:
$$f ( Δ ) = \frac{2 \times S}{1 + P} - S .$$

12. The method of claim 11, wherein the approximation P for the term 2^{-Δ} is based on a Taylor series.

13. The method of claim 11, wherein the approximation P for the term 2^{-Δ} is based on a Maclaurin series.

14. The method of claim 11, wherein the approximation P is defined as follows:
$$if - Δ \geq 0 , then P = 1 + {\sum }_{n = 1}^{N} \frac{{( k \times ( - Δ ) )}^{n}}{n !} and / or$$
$$if - Δ < 0 , then P = \frac{1}{1 + {\sum }_{n = 1}^{N} \frac{{( - k \times ( - Δ ) )}^{n}}{n !}} ;$$
where N is a predetermined order of the series expansion and k is a predetermined factor.

15. A control unit, wherein the unit comprises elements which are configured to carry out the method of claim 1.

16. A pulse oximeter, wherein the pulse oximeter comprises:
a light source;
a light sensor configured to convert a light component transmitted and/or reflected by a body part on irradiation with light from the light source into a sensor signal;
the control unit of claim 15.

17. A computer program, wherein the computer program comprises commands which cause a processor to carry out the method of claim 1 when the processor executes the computer program.

18. A computer-readable medium on which the computer program of claim 17 is stored.
